(19) United States
(12) Patent Application Publication
(10) Pub. No.: US 2008/0161644 A1
(43) Pub. Date: Jul. 3, 2008

(54) METHOD OF AND APPARATUS FOR ATTACHING AN INSTRUMENT TO AN ORGAN WALL

(76) Inventor: Ragae M. Ghabrial, Cincinnati, OH (US)

Correspondence Address: KING & SCHICKLI, PLLC, 247 NORTH BROADWAY, LEXINGTON, KY, 40507, US

(21) Appl. No.: 11/617,812

(22) Filed: Dec. 29, 2006

(51) Int. Cl. A61B 1/00

(52) U.S. Cl. 600/114

(57) ABSTRACT

A method and apparatus for attaching an instrument to an organ utilizes a plurality of protrusions at the end of an overtube for mechanically engaging the end of an overtube to an organ wall. The apparatus, which is particularly suitable for transluminal surgery through a wall of the digestive tract to access the peritoneal cavity, secures the end of an overtube to the organ wall, such as the stomach or colon, to isolate a portion of the organ wall through which an incision for access to the peritoneal cavity is made.

### FIELD OF THE INVENTION

The present invention relates generally to surgery, and more particularly to endoscopic surgery. The invention will be specifically disclosed in a connection with an apparatus for use in transluminal endoscopic surgery, but the methods of the invention and devices constructed in accordance with the principles of the invention can be used in a wider variety of applications.

### BACKGROUND

In recent years, substantial advancements have occurred in abdominal surgery by accessing organs located in the peritoneal cavity through the digestive tract. An endoscope is passed through a natural orifice of the body, such as the mouth or anus, and the endoscope is extended into a selected area of the digestive tract, such as the stomach or colon, that is proximally located relative to the abdominal structure of interest. A luminal incision is then made in wall of the stomach or colon, and an endoscope is then passed through the luminal incision to perform diagnostic or therapeutic interventions on a structure of interest located in the peritoneal cavity.

One potential problem with accessing the peritoneal cavity through the digestive system is the possibility of carrying contaminants from the digestive tract into the peritoneal cavity on the instruments that are inserted through the luminal incision of the wall of the colon or stomach. It is, of course, highly desirable to avoid contamination of the peritoneal cavity, and to perform the diagnostic or therapeutic procedure in a sterile field. One method of reducing the risk of contamination is to use an overtube, that is, a tubular member positioned on the outside of the endoscope through which the endoscope may be is slidably movable. An open end of the overtube is secured to wall of the stomach or colon, and a luminal incision is performed inside the area defined by the overtube. The walls of the overtube then function to isolate the area in which the luminal incision is made from the remainder of the digestive tract. With the end of the overtube secured to the wall of the digestive tract, an endoscope is then extended through the end of the overtube and into the peritoneal cavity through the luminal insertion. With the endoscope inserted into the peritoneal cavity, operational instruments are then passed through a working channel in the endoscope to access to an organ of interest located in the peritoneal cavity upon which a diagnostic or therapeutic intervention is desired.

One way of securing the end of the overtube to the wall of the stomach or colon is through the use of a vacuum. Unfortunately, securing the end of an overtube to the wall of a stomach or colon with a vacuum is not always fully reliable. The stomach wall, for example, is very flexible, and the seal between the end of the overtube and the stomach wall is easily lost whenever the stomach flexes or otherwise moves. When the seal between the end of the overtube and the stomach wall is lost, the luminal incision is no longer isolated from the remaining areas of the digestive track, and the passage of contamination through the luminal incision into the peritoneal cavity may occur. As a consequence, the sterility of the field in the peritoneal cavity in which the diagnostic or therapeutic intervention is occurring is compromised.

### BRIEF SUMMARY

One example of a device utilizing the principles of the invention includes an elongated overtube having a proximal end for location externally of a patient and a distal end for insertion into a lumen of a patient. The overtube has a centrally disposed passage extending from the proximal end to the distal end for permitting the passage of the passage of an endoscope. A tissue engaging structure is positioned on the distal end of the overtube and includes a plurality of protrusions disposed on the distal end of the overtube for mechanically engaging and securing a wall of an organ of a patient. A drive mechanism is provided for selectively moving the tissue engaging mechanism. The drive mechanism is operative to drive the plurality of protrusions into a wall of all organ and to secure the distal end of the overtube to an organ wall.

In one exemplary form of the invention, the drive mechanism rotatably moves the tissue engaging structure to engage an organ wall.

In another exemplary form of the invention, the plurality of protrusions includes protrusions extending in opposite circumferential directions whereby the protrusions extending in each of circumferential directions prevent relative rotational movement between the overtube and the organ wall in the opposite circumferential directions.

In another example, the plurality of protrusions are circumferentially disposed about the centrally disposed passage at the distal end of the overtube.

In another example, the tissue engaging structure includes a pair of counter-rotating tubes at the distal end of the overtube.

In another exemplary form, the plurality of protrusions are disposed on the end surfaces of the pair of counter-rotating tubes, the protrusions on the end surfaces of one of the counter-rotating tubes extending in a first circumferential direction with the protrusions on the in surface of the other of the counter-rotating tubes extending in a second, opposite circumferential direction.

In another exemplary form, the drive mechanism simultaneously rotates the counter-rotating tubes in opposite circumferential directions in time relationship to each other.

In one optional form of the invention, a rupturable seal is provided in the centrally disposed passage in proximity to the distal end of the overtube for preventing contaminants from entering the centrally disposed passage. The seal preferably is formed of transparent material.

Yet another example of the invention is a method of performing transluminal surgery. A first end of an overtube with an interior passage is directed into the digestive tract of a patient, and the first end is directed against a selected organ wall of the digestive tract. The first end is mechanically engaged and interconnected to the wall of the selected organ with tissue engaging structure located proximal to the first end of the overtube. With the first end of the overtube mechanically interconnected to the organ wall, an incision is made through the organ wall, and an endoscope is directed through the interior passage of the overtube into the peritoneal cavity through the incision. A surgical intervention is then performed on an organ in the peritoneal cavity.

The foregoing brief description of certain examples of the invention should not be used to limit the scope of the present invention. Other examples, features, aspects, and embodiments, and advantages of the invention will become apparent to those skilled in the art from the following description, which is by way of illustration, one of the best loads contemplated for carrying out the invention. As will be realized, the invention is capable of other different and obvious aspects, all without departing from the invention. Accordingly, the drawings and descriptions you should be regarded as illustrative the nature and not restrictive.

### BRIEF DESCRIPTION OF THE DRAWINGS

While the specification concludes with claims which particularly point out and distinctly claim the invention, it is believed the present invention will be better understood from the following description taken in conjunction with the accompanying drawings, in which like reference numbers identify the same elements in which:

FIG. 1 is a schematic view of a human stomach with the distal end of an overtube partially inserted into the stomach;

FIG. 2 is a perspective view of the overtube of FIG. 1 showing an exemplary tissue engaging structure at the distal end and an exemplary driving mechanism at the proximal end for rotating tubular components of overtube;

FIG. 3 is an enlarged perspective view of the end portion of the overtube of FIG. 2 showing one exemplary tissue engaging structure in greater detail;

FIG. 4 is fragmentary perspective view illustrating the exemplary drive mechanism shown in FIGS. 2 and 3 in greater detail;

FIG. 5 is a schematic view illustrating the distal end of the overtube of FIGS. 1-4 engaging a wall of the digestive tract; and

FIG. 6 is a perspective view of the endoscope of FIGS. 1-5 schematically showing the overtube in fluid communication with a vacuum source.

Reference will now be made in detail to certain exemplary embodiments of the invention, examples of which are illustrated in the accompanying drawings.

### DETAILED DESCRIPTION OF EXEMPLARY EMBODIMENTS

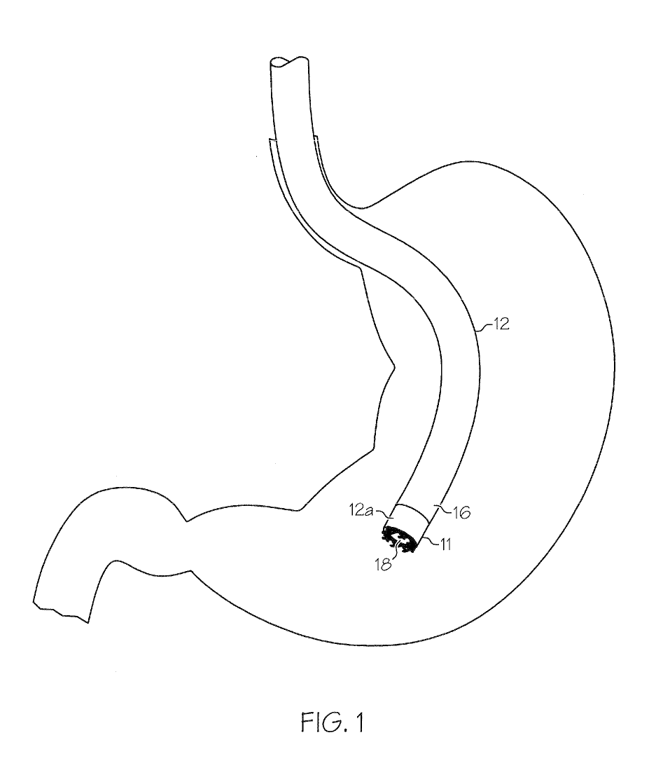

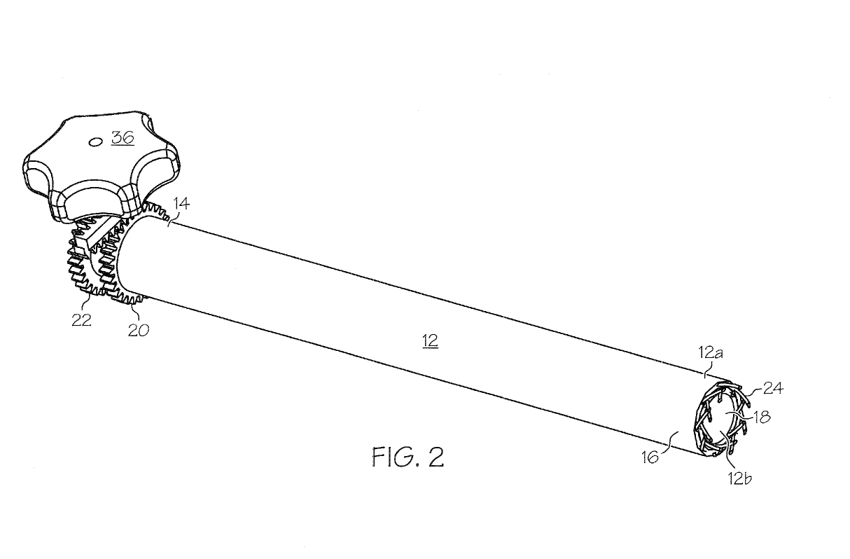

Referring now to the drawings, FIG. 1 schematically illustrates a stomach 10 of a patient into which the distal end 16 of a flexible overtube 12 has been partially inserted. The overtube 12 is formed of highly flexible biocompatible material that slides through the digestive tract with a minimum of frictional resistance. It has an elongated, generally tubular configuration with a proximal end 14 (see FIG. 2) located externally of the patient and a distal end 16 (as illustrated in FIG. 1) designed to be inserted into the digestive tract of a patient. In the illustration of FIG. 1, the distal end 16 of the overtube 12 is disposed in the patient's stomach. As those skilled in the art will readily appreciate, the overtube 12 provides a tubular passageway through which an endoscope or other instrumentation may be directed into the patient's body. In the specific illustration of FIG. 1, the overtube 12 has accessed the stomach 10 through the mouth of a patient. Depending upon the location of the specific portion of the digestive tract on which a diagnostic or therapeutic intervention is desired, access to the location may be made through alternative paths. For example, for a surgical intervention in the lower colon, or in a portion of the peritoneal cavity more closely positioned to the lower colon, access through the patient's anus may be preferable.

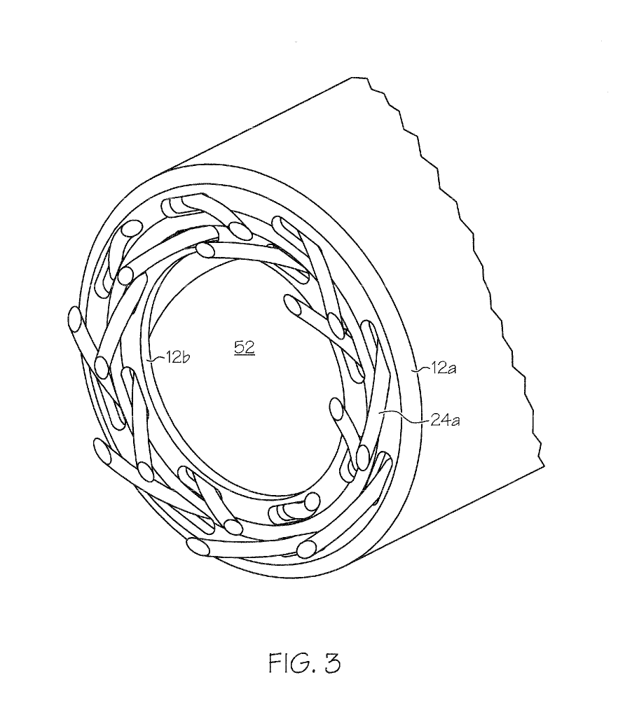

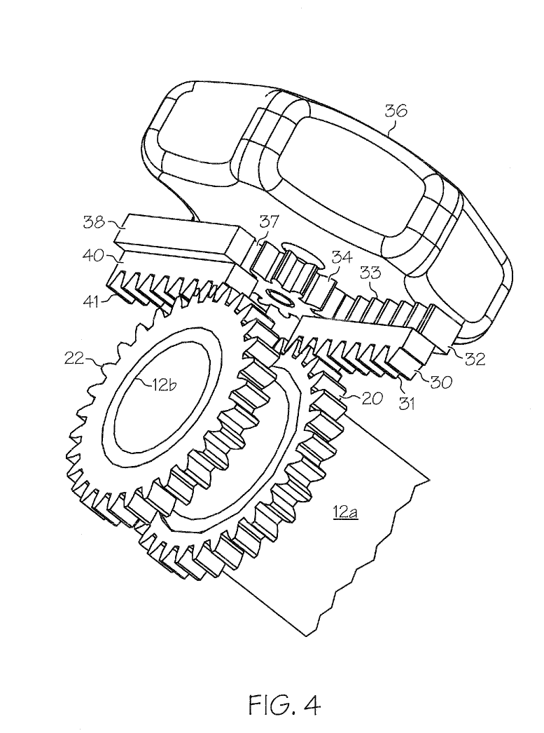

The overtube 12 includes a centrally disposed tubular passage 18 extending from the proximal end 14 to the distal end 16 into which an endoscope or other instrumentation may be slidably inserted. In the specifically illustrated exemplary embodiment, the overtube 12 includes a pair of relatively movable concentrically disposed tubular components 12a and 12b (see FIG. 3) respectfully driven by spur gears 20 and 22 (see FIGS. 2 and 4) located on the proximals ends of the tubular overtube components 12a and 12b respectively.

The distal end of the overtube 12 includes a tissue engaging structure, generally designated by the reference number 24. As more clearly shown in FIG. 3 and explained in greater detail below, the specifically illustrated exemplary tissue engaging structure includes a plurality of needle-like bi-directionally extending projections for mechanically engaging and securing the distal end of the overtube 12 to a wall of an organ of a patient. In the specific exemplary embodiment illustrated in FIG. 3, the plurality of projections are attached to tubular components 12a and 12b, with the projections 24a attached to tubular 10 component 12a projecting in a first circumferential direction and the projections 24b attached to tubular component 12b extending in a second, opposite circumferential direction. This bi-directional configuration of projections provides anti-slip functionality to the tissue engaging structure.

The specifically illustrated drive mechanism for rotating tubular components 12a and 12b is more clearly shown in FIG. 4. As noted above, spur gear 20 is mounted to the outer peripheral surface of the proximal end of tubular component 12a. This spur gear 20 it is driven by a rack gear 30 which rack gear 30 is rigidly attached to a rack gear 32 having meshing teeth 33 orthogonally oriented with respect to the teeth 31 of the rack gear 30. The rack gear 32 is, in turn, meshes with and is driven by a spur gear 34 attached to a manually rotatable knob 36. The spur gear 34 also meshes with and engages teeth 37 of a rack gear 38 located on the opposite side of spur gear 34 from rack gear 32. The rack gear 38 is rigidly attached to another rack gear 40, which rack gear 40 includes teeth 41 which drivingly mesh with and engage the spur gear 22. The spur gear 22 is rigidly attached to the outer periphery of tubular component 12b. Like the relationship between rack gears 30 and 32, the teeth 37 rack gear 38 and the teeth 41 of rack gear 40 are orthogonally oriented with respect to each other. As will be apparent from the illustration of FIG. 4, rotation of the manual knob 36 effectuates counter-rotation of the tubular components 12a and 12b through the above-described gear arrangement.

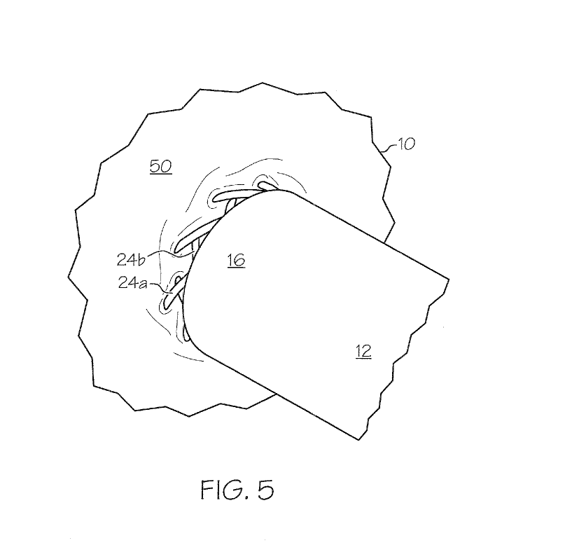

FIG. 5 shows the tissue engaging projections 24 of the specifically illustrated exemplary embodiment located on the distal end of tubular components 12a and 12b as those projections 24 engage a wall 50 of a patient's organ, such as the flexible wall of stomach 10. When the distal end of overtube 12 is moved into contact with an organ wall 50, such as the flexible wall of stomach 10, the manual knob 36 is rotated. This rotation of the manual knob 36 imparts a corresponding rotation to spur gear 34, which, in turn, imparts rectilinear movement of rack gears 30 and 32 in opposite directions. This movement of the rack gears 30 and 32 results in counter-rotational movement of spur gears 20 and 22 which, in turn, effectuates counter-rotational movement of the tissue engaging projections 24a and 24b at the distal ends of tubular components 12a and 12b respectively.

As illustrated in FIG. 5, counter-rotation of the tissue engaging projections 24a and 24b causes such projections to engage and grasp the organ wall 50. Having a configuration with tissue engaging members 24 that move in opposite directions, as in the exemplary embodiment illustrated in the drawings, while optional, is particularly advantageous for securing flexible tissue, such as exists in the stomach. The tissue engaging structure 24a moving in one direction prevents movement of the tissue wall 50 from the tissue engaging structure 24b moving in the opposite direction.

The geometry of the tissue engaging structure 24 also is optional. In selecting the geometry of the tissue engaging structure 24, it obviously is desirable to select a configuration that will reliably secure the distal end of the overtube 12 to the wall of the colon or stomach while simultaneously minimizing any damage to the tissue wall 50. In the exemplary embodiment illustrated, the tissue engaging structure 24 is configured with bi-directional needle-like projections 24a and 24b formed of a shape memory material, such as, for example, a nickel titanium alloy generally known by the acronym NITINOL (an acronym for Nickel Titanium Naval Ordinance Laboratory). As specifically illustrated, the projections 24a of the tissue engaging structure extend from the distal end of tubular components 12a in a direction that is circumferentially opposite from the direction of the projections 24b extending from the distal end of tubular component 12b. As those skilled in the art will appreciate, other configurations for the tissue engaging structure may be used in accordance with the principles of the invention. For example, configurations other than the needle-like configurations illustrated may be used, and, if bi-directional structures are used to provide the tissue engaging structure with anti-slip characteristics, projections in opposite directions can extend from the same tubular component.

Referring once again to FIG. 3, the overtube 12 optionally includes a transparent plastic film 52 in the tubular passage 18 near the distal end 16 of the overtube 12. The plastic film 52 is sealingly engaged to the interior surface of tubular passage 18 in the tubular component 12b to protect an endoscope contained within the overtube 12 from contamination as the overtube 12 is moved through the digestive tract. The plastic film 52 preferably is transparent to allow the passage of light emanating from and reflected to an endoscope positioned on the proximal side of the transparent plastic film 52. This allows a physician using an endoscope located within the tubular passage 18 of the overtube 12 to have visual access to the digestive tract while the endoscope is sealingly isolated from contaminants. The film 52 is ruptured once the distal end of the overtube 12 is secured to the organ wall 50 to permit an endoscope contained within the tubular passage 18 to extend out of the distal end of the overtube 12.

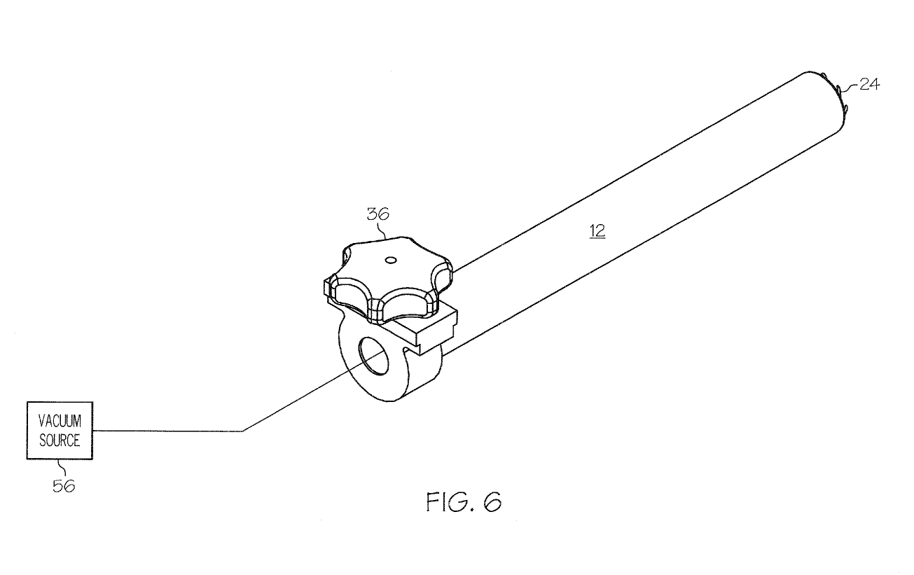

The tissue engaging structure described above can be used as the sole mechanism for securing the distal end of the overtube 12 to the organ wall 50, or it can be used in conjunction with a vacuum assist. Referring to FIG. 6, the overtube 12 is the communication with a vacuum source 56 located externally of the patient, as for example, a vacuum port in a typical operating room. More specifically, the vacuum source 56 is in fluid communication with, and applies a negative pressure through the tubular passage 18 to the distal end of the overtube 12.

In use, the overtube 12 is introduced into the digestive tract, as for example through a patient's mouth or anus, and the distal end of the overtube is moved along the digestive tract until it is optimally positioned relative to the structure in the peritoneal cavity upon which a diagnostic or therapeutic intervention is desired. The distal end of the overtube 12 is then moved against the wall of the digestive tract, as illustrated in FIG. 5, and the knob 36 is rotated, thereby effectuating counterrotation of the tubular components 12a and 12b. This, in turn, rotates the needle-like projections 24 on tubular components in opposite circumferential directions to secure the wall of the digestive tract. With the distal end of the overtube 12 mechanically secured to the digestive tract wall by the tissue engaging structure 24 (and if a plastic film 52 is used, the plastic film 52 is ruptured), a vacuum may then be drawn through the tubular passage 18 to enhance the sealing engagement between the distal end of overtube 12 and the wall 50 of the digestive tract, more fully isolating the portion of the digestive tract wall 50 circumscribed by the end of overtube 12 from the remainder of the digestive tract. An endoscope is moved to the end of the overtube 12, and a cutting instrument (from a working channel of the endoscope) is then used to make an incision through the digestive tract wall 50 in the isolated portion of wall 50 defined by the sealed overtube 12. With the distal end 16 of the overttube 12 sealed and secured against the organ wall 50, the endoscope is slid out of the opening at the distal end 16 of the overtube 12 through the incision and into the peritoneal cavity to perform the desired diagnostic or therapeutic intervention. Once the procedure on the organ in the peritoneal cavity is completed, the endoscope is withdrawn back into the overtube, and the incision is closed in a conventional fashion through instrumentation inserted into a working channel of the endoscope. Once the incision is closed, the knob 36 is rotated to release the digestive tract wall and the vacuum applied through the overtube 12 is discontinued. The overtube 12 and endoscope are then removed from the digestive tract of the patient.

The device disclosed herein can be designed to be disposed of after a single use, or it can be designed to be used multiple times. In either case, however, the device can be reconditioned for reuse after at least one use. Reconditioning can include any combination of steps of disassembly of the device followed by cleaning or replacement of particular pieces, and subsequent reassembly. In particular, the device can be disassembled, and any number of particular pieces or parts of the device can be selectively replaced or removed in any combination. Upon cleaning and/or replacement of particular ports, the device can be reassembled for subsequent use either at a reconditioning facility, or by a surgical team immediately prior to a surgical procedure. Those skilled in the art will appreciate that reconditioning of a device can utilize a variety of techniques for disassembly, cleaning/replacement, and reassembly. Use of such techniques, and the resulting reconditioned device, are all within the scope of the present invention.

The foregoing description of preferred embodiments of the invention has been presented for purpose of illustration and description. It is not intended to be exhaustive or to limit the invention to the precise form disclosed. Obvious modifications or variations are possible in light of the above teachings. The embodiments were chosen and described in order to best illustrate the principles of the invention and its practical applications to thereby enable one of ordinary skill in the art to best utilize the invention in various embodiments and with various modifications as are suited to the particular use contemplated. It is intended that the scope of the invention be defined by the claims appended hereto.

What is claimed is:

1. A device for attaching to and isolating a portion of a wall of an organ of a patient during an endoscopic procedure, comprising:
an elongated overtube, the overtube having a proximal end for location externally of a patient and a distal end for insertion into a lumen of a patient, the overtube having a centrally disposed passage extending from the proximal end to the distal end for permitting the passage of an endoscope;
a tissue engaging structure positioned on the distal end of the overtube, the tissue engaging structure including a plurality of protrusions disposed on the distal end of the overtube for mechanically engaging and securing a wall of an organ of a patient; and
a drive mechanism for selectively moving the tissue engaging structure, the drive mechanism being operative to drive the plurality of protrusions into a wall of an organ and to secure the distal end of the overtube to an organ wall.

2. A device as recited in claim 1 wherein the drive mechanism rotatably moves the tissue engaging structure to engage an organ wall.

3. A device as recited in claim 2 wherein the plurality of protrusions includes protrusions extending in opposite circumferential directions whereby the protrusions extending in each of circumferential directions prevent relative rotational movement between the overtube and the organ wall in the opposite circumferential directions.

4. A device as recited in claim 2 wherein of the plurality of protrusions are circumferentially disposed about the centrally disposed passage at the distal end of the overtube.

5. A device as recited in claim 2 wherein the tissue engaging structure includes a pair of counter-rotating tubes at the distal end of the overtube.

6. A device as recited in claim 5 wherein the plurality of protrusions are disposed on the end surfaces of the pair of counter-rotating tubes, the protrusions on the end surfaces of one of the counter-rotating tubes extending in a first circumferential direction with the protrusions on the in surface of the other of the counter-rotating tubes extending in a second, opposite circumferential direction.

7. A device as recited in claim 6 wherein the drive mechanism simultaneously rotates the counter-rotating tubes in opposite circumferential directions.

8. A device as recited in claim 7 wherein the drive mechanism rotates the counter-rotating tubes in time relationship to each other.

9. A device as recited in claim 1 further including a rupturable seal in the centrally disposed passage for preventing contaminants from entering the centrally disposed passage.

10. A device as recited in claim 9 wherein the seal is located in proximity to the distal end of the overtube.

11. A device as recited in claim 10 wherein the seal is formed of transparent material.

12. A device for attaching to and isolating a portion of a wall of an organ of a patient during an endoscopic procedure, comprising:
an elongated overtube, the overtube having a proximal end for location externally of a patient and a distal end for insertion into a lumen of a patient, the overtube having a centrally disposed cavity extending from the proximal end to the distal end for permitting the passage of an endoscope;
a plurality of multi-directionally extending protrusions extending from the distal end of the overtube, the protrusions being operative to mechanically engage and secure the distal end of the overtube to a wall of an organ of a patient; and
a drive mechanism for selectively moving the t protrusions into a wall of an organ to secure the distal end of the overtube to an organ wall.

13. A device as recited in claim 12 wherein the multi-directional protrusions are configured to prevent slippage between the protrusions and an organ wall.

14. A device as recited in claim 13 further including a mechanism for manually effectuating movement of the protrusions relative to the overtube to selectively engage an organ wall.

15. A device as recited in claim 14 wherein the mechanism for manually effectuating movement of the protrusions is located at the proximal end of the overtube.

16. A method of performing transluminal surgery, comprising the steps of:
a) directing a first end of an overtube with an interior passage into the digestive tract of a patient;
b) directing the first end of the overtube against a selected organ wall of the digestive tract;
c) mechanically engaging and interconnecting the first end of the overtube to the wall of the selected organ with tissue engaging structure located proximal to the first end of the overtube;
d) with the first end of the overtube mechanically interconnected to the organ wall, making an incision through the organ wall;
e) directing an endoscope through the interior passage of the overtube into the peritoneal cavity through the incision; and
f) performing a surgical intervention on an organ in the peritoneal cavity.

17. A method as recited in claim 16 further including the step of:
a) retracting the endoscope into the overtube to remove the endoscope from the peritoneal cavity;
b) repairing the incision; and
c) disengaging the interconnection between the first end of the overtube and the selected organ wall.

18. A method as recited in claim 17 wherein the step of mechanically engaging the first end of the overtube to the wall of the selected organ is achieved by rotating the tissue engaging structure.

19. A method as recited in claim 17 wherein the step of mechanically engaging the first end of the overtube to the wall of the selected organ is achieved by impairing counter-rotation to bi-directionally extending protruding structures located on the end of the overtube.
